(19) United States
(12) Patent Application Publication
(10) Pub. No.: US 2025/0185948 A1
(43) Pub. Date: Jun. 12, 2025

(54) WEARABLE APTAMER MICRONEEDLE PATCH FOR CONTINUOUS MINIMALLY-INVASIVE BIOMONITORING

(71) Applicant: THE REGENTS OF THE UNIVERSITY OF CALIFORNIA, Oakland, CA (US)

(72) Inventors: Sam EMAMINEJAD, Los Angeles, CA (US); Shuyu LIN, Los Angeles, CA (US); Jialun ZHU, Los Angeles, CA (US); Xuanbing CHENG, Los Angeles, CA (US)

(73) Assignee: THE REGENTS OF THE UNIVERSITY OF CALIFORNIA, Oakland, CA (US)

(21) Appl. No.: 18/844,359

(22) PCT Filed: Mar. 6, 2023
(86) PCT No.: PCT/US2023/014654

§ 371 Date: Sep. 5, 2024

Related U.S. Application Data
(60) Provisional application No. 63/317,501, filed on Mar. 7, 2022.
(60) Provisional application No. 63/317,503, filed on Mar. 7, 2022.

(51) Int. Cl. A61B 5/145; A61B 5/00; A61B 5/1473; G16H 20/10

(52) U.S. Cl. CPC A61B 5/14514; A61B 5/14735; A61B 5/4845; A61B 5/685; G16H 20/10; A61B 2560/0462; A61B 2562/028; A61B 2562/125

(57) ABSTRACT

The present embodiments relate generally to an aptamer microneedle patch (“AMPatch”) for providing an example approach to wearable therapeutic drug monitoring (TDM). For example, some embodiments relate to a simple and low-cost EAB-on-microneedle fabrication scheme to develop an AMPatch for in-situ ISF biomonitoring. In some embodiments, a fabrication scheme centers on engineering a gold nanoparticle (AuNP) coating via a single deposition step, which uniquely transforms a clinically-validated needle into a high-quality gold working electrode substrate for strong and compact aptamer immobilization.

### CROSS-REFERENCE TO RELATED APPLICATIONS

The present application claims priority to U.S. Provisional Patent Application No. 63/317,501 filed Mar. 7, 2022 and U.S. Provisional Patent Application No. 63/317,503 filed on Mar. 7, 2022, the contents of which are incorporated herein by reference in their entirety.

### STATEMENT OF GOVERNMENT SPONSORED RESEARCH

This invention was made with government support under 1847729, awarded by the National Science Foundation. The government has certain rights in the invention.

### TECHNICAL FIELD

The present embodiments relate generally to pharmacotherapy, and more particularly to an example approach to therapeutic drug monitoring (TDM) using a wearable microneedle patch.

### BACKGROUND

To realize personalized medicine and effective pharmacotherapy, the right drug needs to be delivered to the right patient at the right dose. In that regard, appropriate dosing for pharmaceuticals that present narrow therapeutic windows, such as antibiotics, is particularly challenging. The high inter-/intra-subject variations—stemming from influential factors including kidney/liver function, tissue penetration, and drug-drug interactions—may often cause the drug level to fall outside the optimal therapeutic window, leading to adverse outcomes (e.g., kidney injury) and ineffective pharmacotherapy.

It is against this technological backdrop that the present Applicant sought a technological solution to these and other problems rooted in this technology.

### SUMMARY

The present embodiments relate generally to an aptamer microneedle patch (“AMPatch”) for providing an example approach to wearable therapeutic drug monitoring (TDM). For example, some embodiments relate to a simple and low-cost EAB-on-microneedle fabrication scheme to develop an AMPatch for in-situ ISF biomonitoring. In some embodiments, a fabrication scheme centers on engineering a gold nanoparticle (AuNP) coating via a single deposition step, which uniquely transforms a clinically-validated needle into a high-quality gold working electrode substrate for strong and compact aptamer immobilization. Following this scheme, the sensing interfaces are built on the tip of shortened acupuncture gold needles, allowing to simultaneously leverage the needles' high sharpness for skin penetration and conductivity for signal routing.

According to certain additional aspects, the present embodiments enable personalized therapeutics by creating a minimally-invasive wearable technology that can be deployed to longitudinally track the pharmacokinetic (PK) profiles of various classes of circulating pharmaceuticals in real-time, thus improving pharmacotherapy outcomes by guiding clinical decisions and facilitating timely interventions.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1A to 1F illustrate aspects of a AMPatch for providing one example of wearable TDM according to embodiments.

FIGS. 2A to 2F illustrate aspects of development and characterization of microneedle aptamer sensors according to embodiments.

FIGS. 3A to 3E illustrate aspects of ex-vivo sensor characterization of an AMPatch according to embodiments.

FIGS. 4A to 4F illustrate aspects of in-vivo characterization in a rat model of an AMPatch according to embodiments.

FIG. 5 illustrates aspects of enabling personalized therapeutics with an example minimally-invasive wearable TDM technology according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

According to certain general aspects, the present Applicant recognizes that to realize personalized medicine and effective pharmacotherapy, the right drug needs to be delivered to the right patient at the right dose. In that regard, appropriate dosing for pharmaceuticals that present narrow therapeutic windows, such as antibiotics, is particularly challenging. The high inter-/intra-subject variations—stemming from influential factors including kidney/liver function, tissue penetration, and drug-drug interactions—may often cause the drug level to fall outside the optimal therapeutic window, leading to adverse outcomes (e.g., kidney injury) and ineffective pharmacotherapy.

To circumvent such issues, patients who are prescribed these medications undergo therapeutic drug monitoring (TDM) sessions. At present, standard practices for conducting TDM involve invasive blood draws, followed by labor-intensive and high-cost lab-based analysis (e.g., chromatography, immunoassay) to capture the drug circulating level at one or two timepoints. Accordingly, the present embodiments relate generally to an aptamer microneedle patch (“AMPatch”) for wearable TDM.

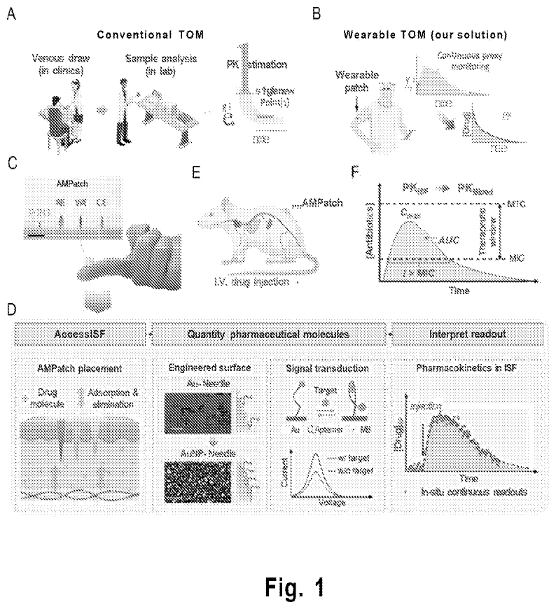

FIGS. 1A to 1F illustrate aspects of a AMPatch for wearable TDM according to embodiments: (A) Schematic illustration of the conventional TDM approach. The approach consists of venous blood draw in clinics followed by analysis in a centralized lab to render single/few drug measurement(s), which is used to estimate the drug's complex pharmacokinetic (PK). (B) Schematic illustration of an envisioned wearable TDM solution. A wearable patch was utilized to render continuous in-situ proxy PK measure (e.g., interstitial fluid (ISF)-based), which is subsequently leveraged to infer the circulating PK. (C) Photos of an assembled AMPatch, consisting of microneedle electrodes inserted in a polydimethylsiloxane (PDMS) substrate. WE, CE, and RE correspondingly denote working, counter, and reference electrodes. Scale bar: 2 mm. (D) Workflow of the AMPatch-enabled wearable TDM solution. (i) Minimally-invasively access drug molecules in dermal ISF using an AMPatch. (ii) The engineered AuNP-μNeedle sensing surface renders compact aptamer binding and the target-aptamer binding event is transduced to the voltammetric readout variations and subsequently enables quantification of pharmaceutical molecules. (iii) In-situ continuous AMPatch readouts are leveraged to construct PK in ISF. (E) Schematic representation of applying an AMPatch to track the PK in a rat model. (F) Application of AMPatch to infer physiological-relevant pharmacokinetic characteristics of antibiotics. MTC and MIC denote minimally toxic and inhibitory concentrations, respectively.

More particularly, as shown in FIG. 1A, the limitations of conventional approaches severely compromise the utility of TDM for optimal dosing in many aspects. Firstly, the turnaround times for results are prolonged, and thus, inadequate to allow for timely intervention. Secondly, the poor temporal resolution of the measurements (mostly confined to a single trough level measurement) inherently limit the accuracy of the current TDM approaches in terms of predicting the drug's highly complex pharmacokinetic (PK) characteristics (e.g., area-under-the-curve (AUC)). Thirdly, because of their limited accessibility, TDM sessions are conducted at sub-optimal rates over the course of treatment, and subsequently, fail to capture longitudinal variations in the drug's PK characteristics. This shortcoming is particularly critical for antibiotics-based treatments, where the antibiotic itself or the co-administered drugs can affect the drug clearance (e.g., as a result of the changes they make to renal function).

Wearable sensing systems targeting non-/minimally-invasively accessible biofluids can in principle address the limitations encountered in current TDM practices by enabling the seamless, continuous, and real-time measurement of the drug levels (FIG. 1B). To this end, probing interstitial fluid (ISF) is a viable approach. A wide panel of pharmaceuticals partition into ISF with high correlation to their circulating levels, many of which may not diffuse into other accessible biofluids such as sweat and saliva. It should be also noted that probing ISF is particularly advantageous in the context of antibiotics-based treatments, given that the ISF compartment is often the intended site for drug reaction, where many bacterial infections start and progress. Therefore, in such cases, the ISF-based measurements may provide a more direct insight as compared to the blood-based counterpart.

Microneedle devices are well-suited to retrieve the molecular information in the ISF. These devices possess sharp, mechanically robust, and short needle-like features that enable easy and fracture-free skin penetration with no/minimal pain. By coupling sensing capability with the microneedle's skin piercing functionality, microneedle devices can be adapted for quantifying analytes in ISF. For this purpose, electrochemical sensing methods are suitable, given that they can render analyte readouts in a sample-to-answer manner and within a compact footprint. However, the demonstrated microneedle devices for in-situ ISF electrochemical sensing rely on enzymes or ionophores for analyte recognition, excluding a wide variety of drug molecules for which these recognition elements are not available.

In that regard, the use of aptamers, as artificially-engineered recognition elements, can greatly expand the library of detectable analytes. By immobilizing redox signal reporter-coupled aptamer molecules onto the surface of an electrode, an electrochemical aptamer biosensor (EAB) can be formed-which can reversibly and in real-time transduce aptamer-target bindings into an electrically measurable signal. A number of EABs have been developed and adapted for the measurement of antibiotics in blood, illustrating the utility of this class of biosensors for complex biofluid analysis. Yet EABs have not been leveraged for in-situ ISF biomonitoring, since their fabrication onto microneedle devices is conventionally considered to be too difficult. To elaborate, for robust EAB-based sensing, a high quality surface (e.g., gold) is needed to ensure the strong covalent binding of the aptamer molecules (typically with the aid of an intermediary thiol group) and the efficient retrieval of the transduced signal. Previously reported sensor-on-microneedle fabrication and integration schemes cannot simultaneously satisfy these sensor-level electrochemical constraints and the device-level structural/mechanical constraints (related to skin penetration). Specifically, the demonstrated microneedle-based sensing devices are limited by the impurity and high surface roughness of their substrates (unsuitable for EAB construction), and are mostly fabricated following complex and costly fabrication schemes.

According to certain aspects, the present embodiments relate to overcoming these and other limitations. For example, some embodiments relate to a simple and low-cost EAB-on-microneedle fabrication scheme to develop an aptamer microneedle patch (“AMPatch”) for in-situ ISF biomonitoring (FIGS. 1C, D). In some embodiments, a fabrication scheme centers on engineering a gold nanoparticle (AuNP) coating via a single deposition step, which uniquely transforms a clinically-validated needle into a high-quality gold working electrode substrate for strong and compact aptamer immobilization. Following this scheme, the sensing interfaces are built on the tip of shortened acupuncture gold needles, allowing to simultaneously leverage the needles' high sharpness for skin penetration and conductivity for signal routing. Illustrating the generalizability of the present approach, developed were multiple microneedle-based EAB devices targeting antibiotics with narrow therapeutic windows (tobramycin and vancomycin) as well as other drugs (doxorubicin: an anticancer drug; thrombin: a procoagulant and anticoagulant). The antibiotics choice is motivated by the fact that their misdosing causes >40,000 acute kidney injuries and >$5B in treatment costs per year.

To illustrate the clinical utility of the presented solution, an AMPatch was specifically configured and deployed to continuously track tobramycin's PK profile in a rat model (FIG. 1E). The analyte choice is motivated by tobramycin's narrow therapeutic window and the high rate of nephrotoxicity incidence in tobramycin treatments (12%), which can be effectively mitigated via advanced TDM solutions. Comparison of the in-situ readouts with the drug's circulating levels revealed the high potential of minimally-invasive ISF measurements for predicting critical circulating PK parameters that are commonly used to guide dosing (FIG. 1F). Overall, the results indicate the suitability of the AMPatch to serve as an advanced yet accessible TDM tool to enable personalized pharmacotherapy.

To construct an AMPatch, some embodiments first fabricate microneedle electrodes by affixing clinically-validated acupuncture needles within a flexible polydimethylsiloxane (PDMS) substrate. This configuration leverages the needles' robustness and sharpness to reliably and painlessly pierce the stratum corneum layer of the skin, making it suitable for accessing dermal ISF analytes. To render real-time and continuous sensing, pharmaceutical-targeting aptamers were immobilized onto the microneedle electrode surface. The distal ends of the aptamers are tagged with redox-active molecules (methylene blue, MB) as signal reporters. The aptamers undergo conformational changes upon binding to the target pharmaceutical, altering the charge transfer rate between the signal reporter and electrode surface, which can be measured via voltammetry-based approaches.

To construct an EAB device, some embodiments include a specifically engineered low-cost AuNP-coated gold microneedle surface (denoted as AuNP-μNeedle). Such an AuNP-μNeedle can serve as the biosensor substrate. This coating is beneficial to the EAB construction and signal transduction as it renders a high-quality surface to simultaneously enable strong aptamer binding (via a self-assembled thiol group) and reliable redox signal retrieval (given its exceptional electrochemical properties). The surface chemistry of the AuNP-μNeedle was first characterized and compared with that of the uncoated microneedle substrate.

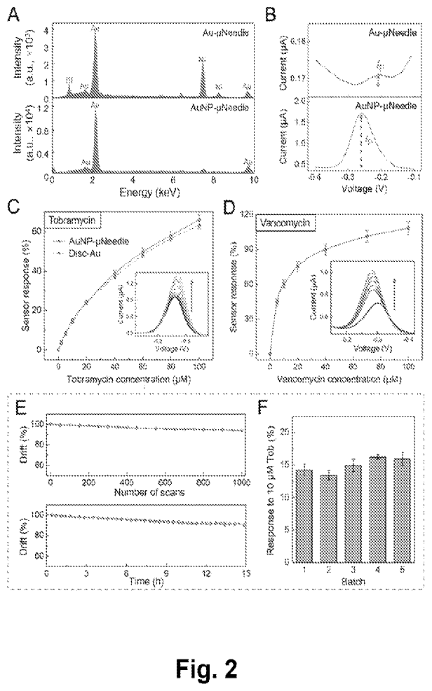

FIGS. 2A to 2F illustrate aspects of development and characterization of microneedle aptamer sensors according to embodiments: (A) EDS spectrum of an Au-μNeedle surface (upper panel) and an AuNP-μNeedle surface (lower panel). (B) Square wave voltammograms of an Au-μNeedle sensor (upper panel) and an AuNP-μNeedle sensor (lower panel) recorded in a blank artificial ISF buffer solution. (C) Au-μNeedle-based and disc Au-based sensors' response to tobramycin. Error bars indicate standard deviations (n=3). Inset shows the corresponding square wave voltammograms acquired from an Au-μNeedle sensor. (D) Readout variations of Au-μNeedle sensors under repetitive interrogation (upper panel) and extended operation time (lower panel). Both experiments were performed in blank artificial ISF buffer solutions. Error bars indicate standard deviations (n=3). (E) Au-μNeedle sensors' response to 10 μM tobramycin. Three sensors were characterized for each batch. Error bars indicate standard deviations. (F) Au-μNeedle-based doxorubicin sensors' response to doxorubicin. Error bars indicate standard deviations (n=3). Inset shows the corresponding square wave voltammograms.

As shown in the scanning electron microscopy-energy dispersive spectroscopy (SEM-EDS, FIG. 2A), a high level of nickel (Ni) impurity was identified on an Au-μNeedle surface, while the AuNP coating effectively suppressed the Ni-associated peaks. Eliminating the Ni exposure is important, because its poor electrochemical performance and associated surface oxide layer can impede the MB signal retrieval and the aptamer immobilization. To further verify this point, representative tobramycin EABs were constructed on both substrates and the biosensors' voltammetric readouts were recorded in a custom-developed artificial ISF buffer environment. As shown in the lower panel of FIG. 2B, for an AuNP-μNeedle biosensor, a well-defined MB reduction peak with a flat baseline was observed, indicating the formation of a compact self-assembled monolayer. However, for an Au-μNeedle biosensor, the voltammetric peak of the signal reporter is difficult to be differentiated from the background (FIG. 2B, upper panel). The AuNP coating is also advantageous for rendering a large effective surface area, increasing the number of packed aptamers on the electrode and subsequently enhancing the signal current and the measurement precision.

To illustrate the generalizability of the approach of embodiments, developed were four microneedle-based EABs (targeting tobramycin, vancomycin, doxorubicin, and thrombin) via immobilizing the corresponding aptamers on the AuNP-μNeedle substrates. As shown in FIGS. 2C, and 2D, for all four cases, the corresponding biosensor responses and analyte concentrations present a monotonic relationship with minimal inter-device variations.

Aligned with the aforementioned motivation of tobramycin PK monitoring, in the subsequent characterization experiments, a primary focus was on tobramycin sensing. Accordingly, first compared was the performance of the microneedle-based tobramycin-EAB with the tobramycin-EAB constructed on a standard gold disc electrode and found that the two possess similar sensitivity levels (FIG. 2C).

Further characterized was the microneedle-based tobramycin-EAB from the standpoint of long-term stability and reproducibility. To investigate the biosensor's long-term stability, recorded was its baseline readout in an artificial ISF buffer under two extreme scenarios: repeated interrogation (1000 scans) and extended operation time (>15 h). In both scenarios, the biosensor readouts presented insignificant drift (<10%, FIG. 2E), indicating that both the electrochemically-driven and time-dependent desorption of the self-assembled monolayer were minimal. The characterization of different batches of the microneedle-based tobramycin-EABs yielded minimal inter-/intra-batch biosensor response variations, demonstrating the high reproducibility of the biosensor fabrication scheme (FIG. 2F).

Further characterized was the microneedle-based tobramycin-EAB using ex-situ models that mimic the envisioned ISF sensing scenario. First, to evaluate the device's continuous sensing capabilities, employed was a phantom gel setup, in which the gels were pre-spiked to different tobramycin concentration levels to represent the analyte concentration variations in dermal ISF. Here, the developed microneedle working electrode was coupled with a silver/silver chloride (Ag/AgCl) reference electrode and a gold counter electrode (both constructed by repurposed solid needles) to form an AMPatch.

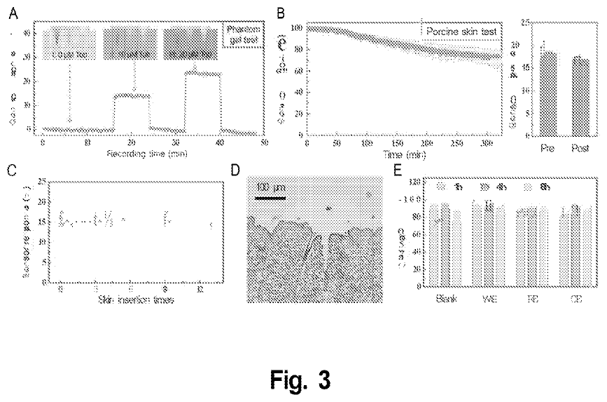

FIGS. 3A to 3E illustrate aspects of ex-vivo sensor characterization of an AMPatch according to embodiments. (A) Continuous measurements of AMPatch sensing response. The AMPatch was inserted into three phantom gels in a rotational manner. Inserts show the schematics of the testing setup (tobramycin concentration level in the phantom gel: 0, 10, and 20 μM). (B) Baseline sensor readouts of Au-μNeedle sensors in a well-hydrated porcine skin tissue. Right: sensors' response to 10 μM tobramycin (in an artificial ISF buffer solution) before and after >300 min skin tissue exposure. The error band and error bars indicate the standard deviation (n=3). (C) Sensors' response to 10 μM tobramycin (in an artificial ISF buffer solution) before and after repetitive insertion into a porcine skin sample. (D) Photo of H&E-stained rat skin showing the penetration of a microneedle electrode. (E) Viability of HDFs cultured with medium exposed to microneedle electrodes for 1, 4, and 8 hours.

More particularly, as shown in FIG. 3A, the AMPatch produced rapid (<1 min) and highly-stable responses to tobramycin in the surrounding environment and the biosensor readout consistently returned to its baseline level in the absence of tobramycin, demonstrating its high suitability for continuous ISF sensing.

Biofouling is one of the main challenges for in-vivo biosensing (including EABs), which can render the sensor unusable within a short period of time. To characterize biofouling in the tissue environment, the developed microneedle-based EABs were inserted into a piece of excised porcine skin and continuously monitored the readouts. FIG. 3B shows that the biosensors maintained 70% of their baseline readouts after >5 h in-skin operation (comparable to previously-demonstrated results) and insignificant changes of the biosensors' response (to 10 μM tobramycin) were observed after the fouling test. An additional biofouling characterization study performed in a protein-spiked buffer solution (with the protein concentration within the physiological range of ISF) revealed smaller drift levels in an extended operation time window (>13 h, FIG. Sx). These results demonstrate that the developed biosensors function properly within its desired duration of operation (transcrouse time frame). Further investigation can explore the source of biosensor drift in the context at hand and drift mitigation approaches should be engineered accordingly.

To characterize the influence of insertion-induced disruption, the biosensors' responses were recorded (in an artificial ISF buffer solution) before and after repetitive insertions into the porcine skin. As shown in FIG. 3C, the sensors' response to 10 M tobramycin remained relatively constant after insertions (˜<10% variations), which can be attributed to the strong adhesion of AuNP to the microneedle substrate as well as the self-assembled monolayer to AuNP.

Prior to deploying the AMPatch for in-vivo pharmaceutical sensing, further investigations were directed to its skin penetration capability and biocompatibility. FIG. 3D shows H&E stained rat skin tissue after microneedle insertion, confirming that the developed microneedle device penetrated the epidermis and accessed the dermal layer of the skin (insertion depth ˜200 μm). To evaluate the biocompatibility of the AMPatch, the human dermal fibroblasts were cultured in the presence of microneedle-based EABs, uncoated needles, and Ag/AgCl needles, which represents impact of the three AMPatch electrodes, respectively. As shown in FIG. 3E, for all the electrodes and examined culturing times, no obvious cell viability change was observed, demonstrating a low cell toxicity for both short-term and relatively long-term device operation.

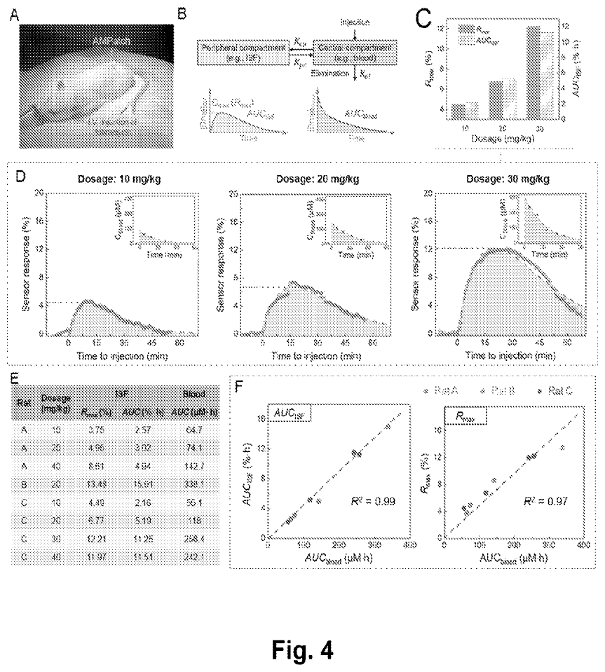

FIGS. 4A to 4F illustrate aspects of in-vivo characterization in a rat model of an AMPatch according to embodiments: (A) Photo of the animal study setup. AMPatch was placed at the back of the rat and the drug was injected intravenously from the tail vein with the aid of a catheter. (B) Schematics of the applied two-compartment model and representative pharmacokinetic profiles in the central and peripheral compartments. Kpc, Kcp, Kel denote the first-order rate constants for distribution, redistribution, and elimination, respectively. (C) ISF PK parameters of one animal (rat C) with three different tobramycin doses. (D) The measured and baseline-corrected AMPatch readouts of FIG. 4C. Insets show the corresponding blood measurements. Dash lines show the fitted PK curves. (E) Tabulated PK results. (F) Correlations between PK parameters in ISF and blood. Left: AUCblood versus AUCISF; right: AUCblood versus Rmax.

More particularly, the developed AMPatch was applied to monitor the PK profile of tobramycin in dermal ISF using a rat model and investigate the drug's ISF-blood correlation. Accordingly, used were three healthy adult Sprague-Dawley rats and the animals were anesthetized during the test (FIG. 4A). An AMPatch was placed at the back of the animal to render continuous ISF monitoring upon intravenous tobramycin injection. No AMPatch-induced bleeding was observed and the skin recovered to its normal state rapidly after AMPatch removal, reaffirming the minimally-invasive nature of the developed technology. To investigate the drug's ISF-blood correlation, blood samples were simultaneously collected before and at intermittent time points after drug injection, followed by standard lab analysis (liquid chromatography with tandem mass spectrometry, LC-MS/MS). The acquired PK readouts were interpreted using a two compartment-based pharmacokinetic model, which captures the drug's distribution (e.g., from blood to ISF) and elimination processes (FIG. 4B). The blood-ISF correlation was evaluated by comparing the drug's circulating AUC (AUC_blood) versus the AUC and the maximum concentrations of ISF drug, where the ISF concentrations can be equivalently presented by the measured sensor response (denoting AUC_ISF and R_max, respectively). The choice of AUC_blood as standard measurement is motivated by the fact that AUC_blood presents the total drug exposure and has been shown to be effective in guiding tobramycin dosing.

An AMPatch according to embodiments was first deployed in a manner to assess its capability of tracking the ISF PK profile in-vivo. After a bolus tobramycin injection (e.g. 20 mg/kg), a rapid increase in sensor readout was observed, followed by a gradual decrease, representing the drug's distribution and redistribution/elimination phases. In a separate control experiment, the injection of a similar amount of saline will not induce changes in the sensor readout. Then, the study was extended to investigate the drug's PK in relation to the injection dosages. To avoid the interference of inter-animal variation, the study was performed on the same rat (with at least 1 week interval between experiments to avoid carry-over effect). As shown in FIGS. 4C and 4D, both R_max and AUC ISF increased with the increase of drug dosages, which was confirmed by the concurrently acquired blood readouts. Similar results were also obtained from another animal. It should be noted that neither the blood readout nor the ISF readout is proportional to the administered dosage, implying the presence of significant intra-animal variation. FIG. 4E tabulates the collective results of all independent experiments (3 animals, 8 trials in total). By comparing results from different animals with the same injection dosage (20 mg/kg), large inter-subject PK variation is observed (AUC_blood: 176+116 μM·h; AUC_ISF: 7.7±5.2%·h). These significant inter-/intra-subject variations are inline with previous observations based on blood analysis and can be attributed to the variations of physiological status (e.g., renal function, weight, diet).

Encouragingly, despite the presence of large PK variations, the captured ISF readouts closely mirror the circulating ones. FIG. 4F shows the high level of correlation between AUC_blood and AUC_ISF (R2=0.99) as well as AUC_blood and R_max (R2=0.97). The result from R_max is of particular interest as it can significantly reduce the time needed to predict the subject's drug exposure (˜20 min for the case of a rat), such that timely intervention can be executed. Collectively, these results demonstrate the high potential of ISF readouts to predict circulating PK.

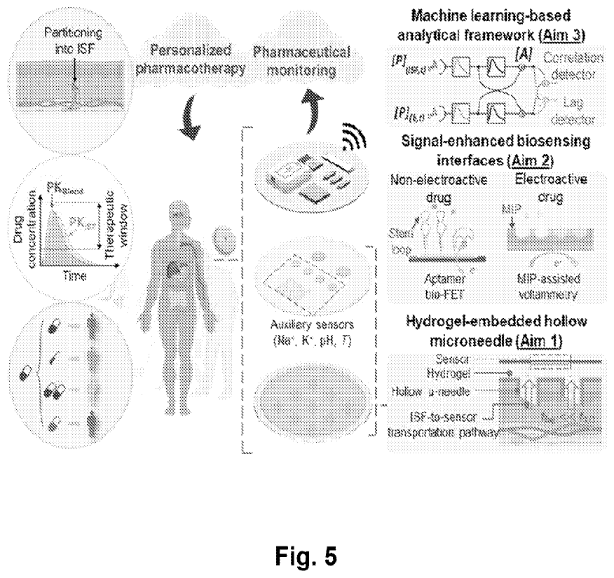

According to certain additional aspects, the present embodiments enable personalized therapeutics by creating a minimally-invasive wearable technology that can be deployed to longitudinally track the pharmacokinetic (PK) profiles of various classes of circulating pharmaceuticals in real-time, thus improving pharmacotherapy outcomes by guiding clinical decisions and facilitating timely interventions. In this way, the present embodiments address a societal grand healthcare challenge: non-optimized medication therapy, which is fueled by inappropriate dosing and patients' poor medication adherence, and results in 275,000 deaths and $530B in healthcare costs, annually (J. H. Watanabe, T. McInnis, J. D. Hirsch, Cost of Prescription Drug-Related Morbidity and Mortality. Ann Pharmacother 52, 829-837 (2018)). Aspects of these and other additional embodiments are shown in FIG. 5.

Currently, medication dosing is based on the drug manufacturer's recommendation, following statistical averages from trials on a relatively small patient sample size. Thus, the prescribed dosage may fall outside the optimal therapeutic concentration window of the individual, resulting in adverse events (e.g., drug toxicity) and ineffective pharmacotherapy. This is particularly critical for patients on antibiotic-, antipsychotic-, anticancer-, antiepileptic-, and post-transplant treatments, for whom the drug dosages must be adjusted such that the corresponding circulating levels fall within oftentimes narrow therapeutic windows. These patients commit to frequent, lengthy, invasive, labor-intensive, and expensive therapeutic drug monitoring (TDM) involving repeated blood draws, with long turnaround times that prohibit adequate and timely intervention. For personalized dose assessment, we require a low-cost, non/minimally-invasive, real-time monitoring modality tracking the medication's PK profile and characteristics (e.g., max concentration: Cₘₐₓ, half-life: t_{1/2}) longitudinally (J. Li, J. Y. Liang, S. J. Laken, R. Langer, G. Traverso, Clinical Opportunities for Continuous Biosensing and Closed-Loop Therapies. Trends Chem 2, 319-340 (2020); H. C. Ates, H. Ceren Ates, J. A. Roberts, J. Lipman, A. E. G. Cass, G. A. Urban, C. Dincer, On-site therapeutic drug monitoring. Trends Biotechnol. 38, 1262-1277 (2020)).

Recent advances in biochemical sensors, device fabrication/integration, and low-power electronics have enabled the realization of wearable systems that can be adapted for tracking circulating molecules in epidermally retrievable biofluids—as most recently demonstrated by a bioanalytical smartwatch (developed by our team) that can measure the concentration of an electroactive pharmaceutical in sweat (S. Lin, W. Yu, B. Wang, Y. Zhao, K. En, J. Zhu, X. Cheng, C. Zhou, H. Lin, Z. Wang, H. Hojaiji, C. Yeung, C. Milla, R. W. Davis, and S. Emaminejad, Noninvasive wearable electroactive pharmaceutical monitoring for personalized therapeutics. P Natl Acad Sci USA 117, 19017-19025 (2020). PMCID: PMC7431025). Here, to establish a generalizable wearable TDM modality for a broad range of pressing clinical applications, we will track pharmaceuticals in interstitial fluid (ISF), which can be minimally—invasively probed. The choice for ISF is ideal, since a wide panel of pharmaceuticals-particularly those with stringent personalized dosing requirements—do not diffuse into biofluids such as sweat or saliva, but partition into ISF with high correlation to their circulating levels (e.g., charged and high molecular-weight pharmaceuticals, e.g. J. Heikenfeld, A. Jajack, B. Feldman, S. W. Granger, S. Gaitonde, G. Begtrup, B. A. Katchman, Accessing analytes in biofluids for peripheral biochemical monitoring. Nat. Biotechnol. 37, 407-419 (2019)).

Accordingly, the present embodiments address the fundamental and intermeshed bottlenecks involved in accessing, quantifying, and interpreting ISF-based pharmaceutical information, by devising convergent innovative device-, sensor-, and data analytics-level solutions: 1) an unprecedented hydrogel-embedded hollow microneedle interface, where the hydrogel simultaneously and uniquely renders an ISF-to-sensor analyte diffusion pathway, a micro-controlled aqueous medium for fouling-resistive sensing, sensor protection, and ease of integration with planar sensors (obviating the need for complex sensor-on-microneedle fabrication); 2) generalizable sensing interfaces—with built-in signal enhancement features—to seamlessly and continuously track electroactive and non-electroactive drugs; and 3) machine learning-based algorithms to mitigate the effect of confounders and to render personalized and predictive estimates of the drug's PK profile. Embodiments include example drugs with narrow therapeutic windows (adjacent table) since their real-time monitoring and personalized dosing are critical to the therapeutic efficacy and prevention of adverse outcomes.

By devising convergent innovative device-, sensor-, and data analytics-level solutions, the present embodiments address the key bottlenecks involved in accessing, quantifying, and interpreting ISF-based pharmaceutical information, to establish an unprecedented minimally-invasive wearable TDM modality. At the device level, embodiments include a hydrogel-embedded hollow microneedle array, where the hydrogel embodiment uniquely renders an analyte diffusion pathway from ISF to sensor and a micro-controlled aqueous medium for sensing. This novel approach also enables ease of sensor integration (via vertical integration with planar sensors, bypassing conventional complex sensor-on-microneedle fabrication) as well as sensor protection (mechanically: at the point of insertion; biochemically: against biofouling (S. Li, J. Dai, M. Zhu, N. Arroyo-Curras, H. Li, Y. Wang, Q. Wang, X. Lou, T. E. Kippin, S. Wang, K. W. Plaxco, H. Li, F. Xia, Hydrogel-coating improves the in-vivo stability of electrochemical aptamer-based biosensors, doi: 10.1101/2020.11.15.383992)).

At the sensor level, embodiments include generalizable sensing interfaces—with built-in signal enhancement features—to seamlessly and continuously track 1) electroactive drugs (here, clozapine), for which signature redox peaks can be exploited for detection and 2) non-electroactive drugs, for which aptamer receptors are available (here, tobramycin and vancomycin). For electroactive drugs, a MIP-assisted voltammetric interface can be included, which synergistically couples a sensitive voltammetric analysis layer and a target-preconcentration, selective, and fouling resistive MIP layer to render reliable electroactive drug detection in the presence of interfering endogenous electroactive species. For non-electroactive drugs, a bio-FET sensing interface can be included, which synergistically couples the aptamer receptors—featuring large, negatively charged DNA stem loop structures—with quasi-2D FET interfaces. This interface renders amplified target binding-induced surface charge perturbation and ultra-sensitive signal transduction, overcoming Debye length limitations (N. Nakatsuka, K. A. Yang, J. M. Abendroth, K. M. Cheung, X. Xu, H. Yang, C. Zhao, B. Zhu, Y. S. Rim, Y. Yang, P. S. Weiss, M. N. Stojanović, A. M. Andrews, Aptamer-field-effect transistors overcome Debye length limitations for small-molecule sensing. Science, 362 (6412), 319-324 (2018)). Both sensors follow Langmuir kinetics: the analyte surface bindings (hence, the sensor response) are dynamic and proportional to the analyte bulk concentration (Nishitani, S. and Sakata, T., Adsorption Isotherm Analysis of a Molecularly Imprinted Polymer Interface for Small-Biomolecule Recognition. ACS Omega 3, 5382-5389 (2018)), as demonstrated by previously reported continuous, regenerable, and real-time aptamer- and MIP-based sensors (N. Arroyo-Curras, J. Somerson, P. A. Vieira, K. L. Ploense, T. E. Kippin, K. W. Plaxco, Real-time measurement of small molecules directly in awake, ambulatory animals. Proc Natl Acad Sci 114, 645-650 (2017); O. Parlak, S. T. Keene, A. Marais, V. F. Curto, A. Salleo, Molecularly selective nanoporous membrane-based wearable organic electrochemical device for noninvasive cortisol sensing. Sci Adv. 4, eaar2904 (2018)).

At the data analytics level, embodiments characterize the PK profile of target drugs in ISF and blood via clinical studies. By augmenting the generated datasets with the state-of-the-art machine learning inference models, a scalable analytical framework can render personalized and predictive estimates of the PK profile of the circulating targets (based on ISF readings). For improved predictive accuracy and to correct for inter-/intra-individual analyte blood-ISF partitioning variability (inherent confounder), embodiments integrate clinically validated/reported sodium (Na+) and potassium (K+) sensing interfaces (W. Gao, S. Emaminejad, H. Y. Y. Nyein, S. Challa, K. V. Chen, A. Peck, H. M. Fahad, H. Ota, H. Shiraki, D. Kiriya, D. H. Lien, G. A. Brooks, R. W. Davis, A. Javey, Fully integrated wearable sensor arrays for multiplexed in situ perspiration analysis. Nature 529, 509-514 (2016). PMCID: PMC4996079) into the sensing system to concurrently track the concentration of these relatively time-invariant endogenous circulating molecules as internal references-normalizing the ISF pharmaceutical readings. Similarly, embodiments include pH and temperature sensors for sensor response calibration. By integrating the microneedle and sensor units with a printed circuit board containing wireless signal acquisition circuitry, a TDM patch is realized (15 g; 4×4 cm2; 8 mm-thick; 50 mW: operable by a compact rechargeable battery, similar to previous wearables (Y. Zhao, B. Wang, H. Hojaiji, Z. Wang, S. Lin, C. Yeung, H. Lin, P. Nguyen, K. L. Chiu, K. Salahi, X. Cheng, J. Tan, B. A. Cerrillos, S. Emaminejad, A wearable freestanding electrochemical sensing system. Sci. Adv. 6, eaaz0007 (2020). PMCID: PMC7083607)). The present patch can monitor the PK profile of one single dose in individuals, creating a foundation for future larger scale and longitudinal studies.

Specific Aim 1: Developing a minimally-invasive hydrogel-embedded hollow microneedle interface to continuously access circulating pharmaceuticals (M1-M24).

Task 1.1: Engineering the hydrogel-embedded hollow microneedle interface. The hydrogel embodiment serves as an analyte diffusion pathway from ISF to sensor (where the quasi-equilibrium analyte concentration in hydrogel is reflective of that in the ISF), and a micro-controlled aqueous media for sensing (S. Y. Lin, B. Wang, Y. C. Zhao, R. Shih, X. B. Cheng, W. Z. Yu, H. Hojaiji, H. S. Lin, C. Hoffman, D. Ly, J. W. Tan, Y. Chen, D. Di Carlo, C. Milla, S. Emaminejad, Natural Perspiration Sampling and in Situ Electrochemical Analysis with Hydrogel Micropatches for User-Identifiable and Wireless Chemo/Biosensing. ACS Sens. 5, 93-102 (2020). PMID: 31786928). The analyte transportation process includes the molecule partitioning from dermal ISF into the hydrogel and molecules diffusion within the hydrogel. The target partitioning is governed by the pharmaceutical/hydrogel polymer chains interactions and the hydrogel water-volume fraction (q) as described by: kᵢ=C_{gel}/C_{ISF}=E_{iφ}, where kᵢ is the partitioning coefficient, Eᵢ is the enhancement factor (capturing the analyte-hydrogel interactions), and C_{gel} and C_{ISF} are respectively the concentrations of the pharmaceutical in hydrogel and ISF. Embodiments synthesize a hydrogel based on 2-hydroxyethyl methacrylate/anionic methacrylic acid (HEMA/MAA) with an optimal composition (a polymer that is approved by the US FDA for the fabrication of contact lens). To promote analyte partitioning into the hydrogel, embodiments optimize the HEMA/MAA ratio and hydrogel pH to increase the electrostatic analyte/hydrogel interactions (for ionized pharmaceutical targets). To enhance the analyte diffusion within the hydrogel structure, embodiments optimize the cross-linking agent (ethylene glycol dimethacrylate) concentration to minimize the physical obstruction and hydrodynamic resistance for diffusion, as well as increase the surrounding mesh amount/size. Also, embodiments adjust the hydrogel water-volume fraction to achieve a relatively high ratio (favorable for both enhanced partitioning/diffusion), while meeting hydrogel stability constraints.

To characterize analyte transport, embodiments use two-photon confocal microscopy and back extraction with UV/Vis spectrophotometry (D. E. Liu, Solute Partitioning and Hindered Diffusion in Hydrogels, p. 1 online resource (2016)). Once established, the hydrogel can be embedded within a hollow microneedle array to continuously and minimally-invasively access ISF. Here, the microneedle array can be fabricated by repurposing the clinically-used hollow needles (e.g., ultra-fine pen needles) via laser-trimming and embedding into a PDMS substrate. Similar approaches have been implemented for prolonged ISF studies of human subjects/animals without report of adverse effects (e.g., skin irritation, discomfort, needle breakage) (P. R. Miller, R. M. Taylor, B. Q. Tran, G. Boyd, T. Glaros, V. H. Chavez, R. Krishnakumar, A. Sinha, K. Poorey, K. P. Williams, S. S. Branda, J. T. Baca, R. Polsky, Extraction and biomolecular analysis of dermal interstitial fluid collected with hollow microneedles. Commun Biol. 1, 173 (2018)). To characterize anti-fouling, the hydrogel-embedded microneedle can be soaked in an artificial ISF solution containing fluorescently-labeled large molecules (e.g., BSA). After removal, the designated sensor location will be imaged to probe for the presence of large molecules. To characterize its mechanical properties, the integrated device can be repetitively inserted into a porcine skin, and the structural integrity of the hydrogel will be examined.

Task 1.2: Validating the biocompatibility and sterility via ex-situ and in-situ characterization studies. Evaluated is the biocompatibility of hydrogel-embedded microneedle array via in-vitro characterization and human subject validation studies. Embodiments first use human dermal fibroblasts, to evaluate the biocompatibility by assessing effects on cell proliferation, apoptosis, senescence, and cell's morphological changes. Then, embodiments can perform biocompatibility validation in-vivo with 10 healthy subjects and 5 patients from each drug group. An example hydrogel-microneedle array according to embodiments is applied against skin following established clinical safety protocol and standard procedures. Safety and tolerability will be determined based on frequency of skin adverse events (AE) occurrence. Both patient reported symptoms (e.g., pain, itching) and images of the site will be recorded to fully document the AE. Skin AEs will be categorized as to their type and severity based on Common Terminology Criteria for Adverse Events (CTCAE, v5.0; s.23. Skin and subcutaneous tissue disorders). Upon review of patient documentation and the severity of the AE, a determination will be made as to the need to withdraw a subject from the study. If this occurs, the use of the device is halted, and its design (materials and geometry) can be revisited to identify the contributing factor to the AE and to devise a suitable alternative. Embodiments use UV sterilization, and if necessary, gamma irradiation, to sterilize the fabricated hydrogel-embedded microneedles. Embodiments can also perform bioburden tests to evaluate the sterility of the assembled device.

Task 1.3: Characterizing the sampling performance of the hydrogel-embedded hollow microneedle via ex-situ and in-situ studies. Embodiments characterize the ISF-based pharmaceutical sampling efficiency of the hydrogel-embedded hollow microneedle by using a custom-developed Franz cell setup and performing human subject testing. The Franz cell setup consists of: 1) a porcine skin layer to mimic the mechanical and analyte transport properties of human skin; 2) a donor chamber underneath the skin layer (containing a pharmaceutical-spiked artificial ISF sample); and 3) a collection chamber on top of the skin layer. The sampled pharmaceutical in the hydrogel will be analyzed by standard lab instruments (e.g., liquid chromatography with tandem mass spectrometry, LC-MS/MS, after extraction into a buffer solution). The equilibrium time for analyte transportation (time required for analyte concentration at the designated sensor location to reach 90% of bulk concentration) will be determined accordingly. For in-situ characterization, 20 patients from each drug group can be used, and ISF sampling can be performed using both the hydrogel-embedded hollow microneedle and standard ISF extraction interfaces (using solid microneedle to penetrate the skin, followed by applying negative pressure (P. P. Samant, M. M. Niedzwiecki, N. Raviele, V. Tran, J. Mena-Lapaix, D. I. Walker, E. I. Felner, D. P. Jones, G. W. Miller, M. R. Prausnitz, Sampling interstitial fluid from human skin using a microneedle patch. Sci. Transl. Med. 12, (2020))) at the proxy spots. The results from the two methods will be compared to examine whether our sampling interface can capture the same trend (among different trials) as the standard interface.

Specific Aim 2: Developing generalizable wearable pharmaceutical sensing interfaces with built-in signal enhancement features (M1-M30).

Task 2.1: Developing a label-free and reagentless bio-FET sensing system. Here, embodiments include a generalizable wearable bio-FET-based sensing methodology to quantify the target drugs (here, tobramycin or vancomycin) with high sensitivity, selectivity, and stability, and in a sample-to-answer manner. To this end, embodiments develop FETs with nanometer-thin film oxide-based channels, then modify them with high-affinity nucleic acid aptamer receptors that feature stem loop structures. Using this configuration, the aptamer functionalized surfaces can translate target binding-induced conformational change (containing large, negatively charged DNA stem loop) into measurable surface charge perturbations (modulating effective gate-source voltage). This example design allows for harnessing the signal amplification effect-from the stem loop-induced surface charge perturbation- and ultra-sensitive signal transduction-rendered by the quasi-two-dimensional FET interface-to overcome the fundamental challenge of the Debye layer screening. This enables highly sensitive detection of target drugs that diffuse in the hydrogel matrix. To construct the proposed sensing interfaces, embodiments leverage and modify (to incorporate the stem-loop feature) the readily reported/validated aptamer sequences. To define the FET's nanometer-thin channel regions, embodiments spin-coat In₂O₃ on a flexible polyimide substrate via solution-processed sol-gel chemistry followed by patterning with reactive ion etching. Preliminary studies already indicated that the proposed bioFET configuration can monitor biomarkers with concentration down to 1 pM (with potentially tunable dynamic detection range).

Task 2.2: Developing a MIP-assisted voltammetry sensing system (targeting electroactive drugs). To detect electroactive drugs (here, clozapine), embodiments can use a MIP-assisted voltammetry sensing interface, featuring a sensitive voltammetric analysis layer as well as a target-preconcentration, selective, and fouling resistive MIP layer. These and other embodiments utilize boron-doped diamond electrodes (BDDE) as the sensor substrate, which features a wide electrochemical potential window, a low background current, an intrinsically high biofouling resistance, operational stability, and biocompatibility. To fabricate the sensor's primary layer (voltammetric analysis), some embodiments include is an optimized carbon nanomaterial dispersed-polymeric layer (e.g., carbon nanotube/graphene/platinum nanoparticles; Nafion/chitosan/gelatin). With this approach, embodiments increase the electron transfer rate, and thus, enhance the sensor's sensitivity. The polymer matrix can also improve the selectivity by tuning the polymer chain/molecule electrostatic interactions. The MIPs can be fabricated using precipitation polymerization. Specifically, the pharmaceutical molecules are used as the template and reacted with MAA, EGDMA, and AIBN (functional monomer, cross-linking agent, and initiator of the imprinting polymerization). The template is removed from the obtained MIPs using methanol. The suspension of template-free MIP is deposited onto the sensing electrode. The MIP layer increases the drug molecule concentration at the electrode surface and prevents the non-target molecules from approaching the electroanalysis layer. This simultaneously enhances the sensor's sensitivity, selectivity, and fouling resistance.

Specific Aim 3: Developing a scalable analytical framework to infer the circulating target's pharmacokinetic profile based on ISF readings (M6-M36).

Task 3.1: Characterizing the targets' PK profiles in ISF and blood. Clinical studies can be performed to track the PK profile of certain targets in ISF and blood after a single-dose administration. The generated datasets from these studies can provide a foundation to develop our proposed predictive models in Task 3.2. 20 patients from each drug group can be included, and blood/ISF sampling will be scheduled around their readily-prescribed administration time windows. Blood samples will be obtained per standard pharmacokinetic individualized assessment protocols (J. Brockmeyer, R. Wise, E. Burgener, C. Milla, A. Frymoyer, Area under the curve achievement of once daily tobramycin in children with cystic fibrosis during clinical care, Pediatr. Pulmonol. 1-8 (2020). PMID: 32827334), and ISF samples can be collected 1 h before and every 30 min after the administration for an extended time window beyond the drug's t_{1/2} (˜4-8 h post-admin). The samples will be analyzed offline following our established assay protocols (LC/MS-MS for target pharmaceuticals, and standard probes for internal references/auxiliary measurements: Na+, K+, pH, temperature) (Id.).

Task 3.2: Developing predictive Bayesian time-series models to infer circulating levels and pharmacokinetics. Embodiments include time-series models to predict the targets' circulating pharmaceutical levels and PK characteristics based on the ISF readings, while accounting for the inter-/intra-individual analyte blood-ISF partitioning variability (inherent confounder). Here, embodiments utilize the generated datasets from Task 3.1, and consider the blood pharmaceutical levels as latent variables observed at specific time-points, and the ISF readings as noisy observations of the blood pharmaceutical levels. To correct for analyte blood-ISF partitioning variability, the concentration profile of relatively time-invariant endogenous circulating molecules (e.g., Na⁺, K⁺, effective measures of blood-ISF partitioning efficiency (Id.)) will be obtained and leveraged as internal reference(s) to normalize the ISF pharmaceuticals readings. Formulating a physiologically-motivated model, we will describe the relationship between the target's concentration levels in blood and ISF as (assuming negligible partitioning/diffusion lag time as compared to t_{1/2}):

$${[ P ]}_{( ISF , i , k )} = {( β + {γ}^{T} {Z}_{i , k} ) [ P ]}_{( B , i , k )} + {ϵ}_{i , k}$$

where [P]_{(ISF,i,k)} and [P]_{(B,i,k)} denote the ISF and blood concentration levels of the target pharmaceuticals P (e.g., tobramycin, vancomycin, or clozapine) in the subject k at time point i. Z_{i,k} denotes the measurements of internal reference (e.g., Na⁺, K⁺), ϵ_{i,k} denotes unmodeled variation, and β and γ denote the parameters of the model. Here, it can be assumed that the latent blood pharmaceutical concentration in subject k at time point i will affect the concentration in this subject at the next time point (i+1). To model this behavior, it is assumed that the latent blood analyte concentrations over time are drawn from an underlying Gaussian process [P]_{(B,t)}˜f(t) that has the property that values at nearby timepoints are highly correlated. Accordingly, embodiments explore functions for which the outputs at two time points (tᵢ,tᵢ₊₁) are correlated as exp(−α(tᵢ₊₁−tᵢ)). The unmodeled noise ϵ_{i,k} is assumed to follow a normal distribution with mean zero and variance σₖ₂. The parameters of the model are (α, σₖ₂, βₖ, γₖ), where (σₖ₂, βₖ, γₖ) are individual-specific parameters. Leveraging the generated datasets, embodiments estimate the parameters by maximizing marginal likelihood and the posterior probability distribution of the parameters by Markov-chain Monte Carlo. The adequacy of the model can be tested by performing model diagnostics and evaluating predictive accuracy on leave-one-out validation. Finally, following the workflow described in Task 3.1, embodiments can randomly select a subset of the patients with an established personalized model, and evaluate the predictive accuracy of our ISF-sensing system and analytical framework to track the PK profile of the circulating target drugs. The estimated target drug levels in blood can be fitted to an established two-compartment model to extract the relevant PK parameters (absorption, distribution, redistribution, and elimination rate constants). The estimated PK parameters can be compared to those extracted from the blood readings, and to those reported in the literature to further evaluate the predictive accuracy of the model.

The outcome is an unprecedented wearable pharmaceutical monitoring technology, with real-time information sensing/transmission capabilities. This can enable innovative patient-centered pharmacotherapy solutions—including drug personalized dosing, remote monitoring, and compliance/abuse monitoring—and make possible a broader range of large-scale drug development investigations. The large datasets to be generated (and subsequent derivative biomonitoring solutions) can be contextualized in relation to Patient Reported Outcomes, and ultimately realize an adaptive and iterative pathway to optimal drug and treatment development. Coupling the present monitoring modality with transdermal drug delivery solutions, will ultimately realize a fully-integrated and feedback-controlled closed-loop technology. The versatility of the sensor, device, and data analytics solutions of embodiments allows for their application to enable monitoring of biomarkers in various biofluids and potentially address numerous other unmet clinical needs. In this way, embodiments address the grand healthcare challenge of precise population-level disease prevention and management.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

1. A device comprising an aptamer microneedle patch (“AMPatch”) configured to provide wearable therapeutic drug monitoring (TDM).

2. The device of claim 1, wherein the AMPatch includes a gold nanoparticle (AuNP) coating on a clinically-validated needle.

3. The device of claim 2, wherein the gold nanoparticle coating configures the needle into a high-quality gold working electrode substrate for strong and compact aptamer immobilization.

4. The device of claim 1, further comprising sensing interfaces built on the tip of shortened acupuncture gold needles.

5. A simple and low-cost EAB-on-microneedle method of fabricating an aptamer microneedle patch (AMPatch) for in-situ ISF biomonitoring, comprising:
engineering a gold nanoparticle (AuNP) coating via a single deposition step, which uniquely transforms a clinically-validated needle into a high-quality gold working electrode substrate for strong and compact aptamer immobilization.

6. The method of claim 5, wherein sensing interfaces are built on the tip of shortened acupuncture gold needles, allowing to simultaneously leverage the needles' high sharpness for skin penetration and conductivity for signal routing.

7. A method using minimally-invasive wearable technology, comprising:
longitudinally tracking the pharmacokinetic (PK) profiles of a drug included in one various classes of circulating pharmaceuticals in real-time using an aptamer microneedle patch (AMPatch), thereby improving pharmacotherapy outcomes by guiding clinical decisions and facilitating timely interventions.

8. The method of claim 7, wherein the AMPatch includes a gold nanoparticle (AuNP) coating on a clinically-validated needle.

9. The method of claim 8, wherein the gold nanoparticle coating configures the needle into a high-quality gold working electrode substrate for strong and compact aptamer immobilization.

10. The method of claim 7, wherein the minimally invasive wearable technology further includes sensing interfaces built on the tip of shortened acupuncture gold needles.

11. The method of claim 7, further comprising providing generalizable wearable pharmaceutical sensing interfaces with built-in signal enhancement features.

12. The method of claim 7, further comprising providing a scalable analytical framework to infer the circulating target's pharmacokinetic profile based on ISF readings.

13. The method of claim 7, wherein the minimally-invasive wearable technology includes a hydrogel-embedded hollow microneedle interface, where the hydrogel simultaneously and uniquely renders an ISF-to-sensor analyte diffusion pathway, a micro-controlled aqueous medium for fouling-resistive sensing, sensor protection, and ease of integration with planar sensors.

14. The method of claim 7, wherein the minimally-invasive wearable technology includes generalizable sensing interfaces with built-in signal enhancement features to continuously track electroactive and non-electroactive drugs.

15. The method of claim 7, further comprising:
using machine learning-based algorithms to mitigate the effect of confounders and to render personalized and predictive estimates of the drug's PK profile.
